(19) United States
(12) Patent Application Publication
(10) Pub. No.: US 2026/0083653 A1
(43) Pub. Date: Mar. 26, 2026

(54) COMPONENT SYSTEM AND METHOD FOR PREPARING A TOOTH WHITENING PREPARATION

(71) Applicant: Cobea AG, Daenikon (CH)

(72) Inventors: Alexander WAELCHLI, Zuerich (CH); Reto WAELCHLI, Zuerich (CH)

(73) Assignee: Cobea AG, Daenikon (CH)

(21) Appl. No.: 19/112,302

(22) PCT Filed: Mar. 13, 2023
(86) PCT No.: PCT/IB2023/052410

§ 371 Date: Mar. 17, 2025

(30) Foreign Application Priority Data
Sep. 23, 2022 (CH) CH001106/2022

(51) Int. Cl. A61K 8/49; A61K 8/04; A61K 8/19; A61K 8/34; A61K 8/365; A61K 8/73; A61K 8/81; A61Q 11/00

(52) U.S. Cl. CPC A61K 8/492; A61K 8/042; A61K 8/19; A61K 8/345; A61K 8/365; A61K 8/73; A61K 8/8152; A61Q 11/00

(57) ABSTRACT

A component system for preparing a physiologically acceptable tooth whitening preparation includes an active ingredient component that includes an organic tooth whitening substance, and an activation component that is designed to adjust a physiologically acceptable pH of the active ingredient component in a range between 7 and 10.5. Further, a stabilized active ingredient component is used in the component system and a method prepares an activated physiologically acceptable tooth whitening preparation.

The present invention relates to a component system for preparing a physiologically acceptable tooth whitening preparation, a corresponding method, and stabilized active ingredient components for use in the stated component system, and use of the component system per se, all according to the preambles of the independent claims.

### TECHNOLOGICAL BACKGROUND

White teeth are an important part of a healthy and well-groomed appearance. Surface stains, which may be formed, for example, by coatings on the teeth such as plaque and/or tartar, may be removed within the scope of a professional teeth cleaning. In medical teeth cleaning processes, plaque and/or tartar are/is mechanically removed by a skilled technician using appropriate tools. However, underlying internal discolorations cannot be whitened with this process. Because great cosmetic importance is placed on white teeth, methods have been developed to further whiten the tooth surface. This type of tooth whitening provides good results, even if a darker tooth color is innate due to genetic predisposition. Such methods used for tooth whitening are referred to as “bleaching.” Methods that are suitable for tooth whitening generally use a more or less strong oxidation agent in order to achieve desired whitening of the teeth. It is essential that the chemically performed tooth whitening is as mild as possible and results in no long-term adverse effects on the health of the teeth and the gums.

Since chemical treatment of the teeth for tooth whitening requires a certain degree of technical expertise, most countries require that tooth whitening must be performed by a specially trained professional. Compounds containing hydrogen peroxide as a bleaching agent for the teeth are often used by dental practices or dental hygiene studios. For cosmetic bleaching and tooth whitening methods, hydrogen peroxide is usually used in the form of carbamide peroxide. In simple terms, hydrogen peroxide penetrates into the tooth and decomposes, with the formation of reactive oxygen which reacts with colorants in the tooth. In the process, the oxygen radicals chemically alter the colorants in such a way that they are no longer discernible. The theoretical basis for use of hydrogen peroxide and its reactivity is described in The Chemistry of Functional Groups, Peroxides (Patei, S. et. al; John Wiley & Sons Ltd. 1983).

For practical application, a rough distinction may be made between methods that are suitable for the end user and home use, and those that are reserved for specially trained personnel. In principle, this distinction may be made primarily based on the reagents used, wherein particularly high concentrations of certain active ingredients, for example peroxide, require use by a professional, while lower concentrations may be suitable for home use. However, the concentration of the active ingredient, in this case hydrogen peroxide, largely determines the effectiveness and sustainability of the tooth whitening. Concentrations of 0.1% hydrogen peroxide are permitted in many countries for agents for home use. Concentrations of up to 38% hydrogen peroxide, for example, may be reserved for dental practitioners, where professional treatment is also necessary for the gums, for example, in order to protect them during the treatment. Additional supporting physical agents, for example, are occasionally used, such as treatment with light in the blue and/or ultraviolet spectrum.

The frequently observed side effects are a particular drawback of tooth bleaching using the known conventional hydrogen peroxides. Thus, for example, increased sensitivity of the teeth may be experienced for several days. The use of tooth whitening agents, which have a pH in the acidic range, is also particularly disadvantageous. These agents may wear down the surface of the tooth enamel or roughen the tooth surface, which promotes penetration of staining substances.

EP 155 683 A1 (Thornthwaite, D. W., et al.) describes an alternative to the commonly used hydrogen peroxide, one that is based on the organic acid phthalimidoperoxycaproic acid (PAP).

PAP is a peroxy acid having good oxidation potential. Without being bound to this theory, the oxidative effect of the peroxy acids appears to be based on the acidity of the O—H bonds and also the electronegativity of the imide group.

A particular advantage of phthalimidoperoxycaproic acid (PAP) is that it achieves an effect comparable to peroxides, but without weakening the tooth enamel or irritating the oral mucosa.

Qin et al. (Qin, J. et al.; A bio-safety tooth-whitening composite gels with novel phthalimide peroxy caproic acid; Composites Communications 13 (2019) 107-111) describe the action of PAP in a polymer gel for use as a tooth whitening mixture. Tooth whitening comparable to that using conventional hydrogen peroxides was achieved; comparatively higher concentrations of PAP were necessary to achieve the analogous effect of hydrogen peroxide tooth whitening.

Since correspondingly high concentrations are in turn less suitable for home use by the end user, there is a need for appropriate point-of-care approaches that enable cosmetic tooth whitening, are easy to use, and avoid at least one of the disadvantages in particular of the conventional point-of-care tooth whitening methods that are based on hydrogen peroxide.

### DESCRIPTION OF THE INVENTION

Therefore, it is an object of the present invention to provide a component system that is easy and reliable to use, and that enables effective tooth whitening in particular in a point-of-care setting and overcomes at least one disadvantage of the known system.

In particular, the aim is to prepare such a component system that may be made ready for use in a few simple steps and requires little training for the technician. Further aims are to provide a corresponding method as well as a stabilized active ingredient component for the component system mentioned at the outset, and use thereof.

At least one of these objects has been achieved with a component system, a stabilized active ingredient component, and/or a method for preparation and/or use of a component system according to the characterizing portion of the independent claims.

One aspect of the present invention relates to a component system for preparing a physiologically acceptable tooth whitening preparation. This is particularly preferably a point-of-care physiologically acceptable tooth whitening preparation.

Within the meaning of the present invention, “point of care” may be understood, for example, as contrary to the prepared mixing at the site of manufacture. Point of care is provided, for example, where the tooth whitening is performed. This may be a medical practice or also home use. In this sense, point of care may be further understood, for example, in that the application is performed by a trained professional. This trained professional is particularly preferably a dental practitioner, a dental hygienist, and/or a dental care assistant. The occupational titles are subject to various requirements, and may vary depending on the country; however, a certain official regulation for practicing the profession and training for use of the chemicals in the method according to the invention are essential. The point of care may, but does not have to, take place in a dental office or a dental studio. There has been a trend recently for dental hygienic services to be offered independently of medical primary care and treatment by dental practitioners, as has occurred in the fields of physical therapy and medical massage. The present invention is particularly suited for use in a dental studio by trained professionals.

Within the meaning of the present invention, detectable lightening of a tooth color may be understood as tooth whitening or bleaching. Effective tooth whitening may be easily checked by use of a reference scale. Before the treatment is started, a certain shade level is assigned to the teeth, using a scale, and after the treatment, is subsequently compared again to the reference scale to objectively determine an improvement, i.e., a change in the degree of whiteness of the teeth. Such scales are known in the practice of dentistry. Within the meaning of the present invention, tooth whitening is achieved when at least one scale degree, for example of a VITA color scale with shades from A1 to D4, has changed in the direction of a lighter value.

The component system according to the invention comprises an active ingredient component that includes an organic tooth whitening substance. The active ingredient component including an organic tooth whitening substance is particularly preferably present with an acidic pH, preferably with a pH below 7. The component system according to the invention further comprises an activation component for adjusting a physiologically acceptable pH of the active ingredient component in a range between 7 and 10.5. The activation component is particularly preferably an alkaline activation component.

In one particular embodiment, the activation component is designed to adjust the active ingredient component to a physiologically acceptable pH between 8.5 and 9.5.

In one particular embodiment, the organic tooth whitening substance is an oxidation agent, in particular an oxidation agent at least in a basic pH environment.

It has surprisingly been found that by use of the present component system, a tooth whitening preparation, in particular a point-of-care tooth whitening preparation, may be provided which overcomes a number of disadvantages. The organic tooth whitening substance is present at a pH below 7, for example. This substance is stable at this pH. However, for application to the teeth an acid is unsuitable, since the tooth is thereby roughened and discoloring substances may once again easily penetrate into the tooth. In addition, use of an acid is generally detrimental to dental health, since the tooth enamel may be affected. However, if the tooth whitening substance is prepared in advance in a physiologically acceptable pH environment, the storage life of the organic tooth whitening substance is reduced significantly. It is assumed that the oxidative effect has diminished after only a few hours, and has completely disappeared after ten days at the most.

By use of the component system according to the invention, it is possible to prepare a physiologically acceptable tooth whitening preparation, containing an organic tooth whitening substance, at the point of use as needed and in the necessary quantity, and to make it directly usable by a professional.

In one particular embodiment, the activation component is pre-dosed, so that with a defined quantity of the activation component it is designed to provide the above-mentioned pH in the organic active ingredient component.

In one particular embodiment, the active ingredient component is accommodated in a first component container. The activation component is preferably accommodated in a second component container. For use, for example a defined quantity of the activation component or the entire contents of the second component container may be transferred into the first component container. After careful mixture of the two components, the tooth whitening preparation is ready for use.

In one particular embodiment, the activation component contains a base in solution with a base constant pKb of 4 maximum. A base is preferably used which is adequate for neutralization of the acidic pH of the active ingredient component and preferably conversion into the alkaline range. Depending on the base strength, a different predefined quantity is necessary to adjust the suitable physiologically acceptable pH of the active ingredient component. A particular advantage of the invention is that operations may be carried out with comparatively much higher concentrations of the organic tooth whitening substance, in particular PAP, resulting in overall more effective and/or sustainable tooth whitening. During use, the correspondingly larger quantity of PAP may be made ready for use by bringing its pH to a physiologically acceptable level, using a base in solution in the form of the activation component. It is not necessary to limit the quantity of organic tooth whitening substance to a pH below 7 for safety reasons, since the application at the point of care ensures that the pH varies in a physiologically acceptable range during use.

In one particular embodiment, the active ingredient component contains at least one caproic acid as an organic tooth whitening substance. This is particularly preferably a peroxy acid, which more preferably is an imidoperoxycarboxylic acid. The organic tooth whitening substance is particularly preferably PAP. One example of a suitable organic tooth whitening substance is 6-(phthalimido) peroxyhexanoic acid, or 6-(1,3-dioxo-1,3-dihydro-2H-isoindol-2-yl) hexaneperoxoic acid according to IUPAC. In the present patent application, the common abbreviation PAP is used for the sake of simplicity to designate all phthalimidoperoxycaproic acids that are suitable for the present patent application.

Alternatively and/or additionally, other acids are suitable. One example of another suitable acid is 4-phthalimidobutyric acid. Use of pthalimidoperoxyheptanoic acid would likewise be conceivable. However, PAP is preferred.

One advantage of PAP is the comparatively good tolerability, even at elevated concentrations. Due to the improved tolerability, the training effort is reduced and the application as a whole is made more reliable.

In one particular embodiment, the organic tooth whitening substance is present in the active ingredient component in a concentration of at least 7%. Within the meaning of the present invention, the percentages in mixtures and solutions are to be understood as percentages by weight in each case. The organic tooth whitening substance is particularly preferably present in the active ingredient component in a concentration of at least 10%, preferably between 10% and 40%, more preferably between 10% and 20%, and particularly preferably approximately 12%.

In one particular embodiment, the active ingredient component additionally includes further auxiliary substances, in particular solvents. The solvent is preferably water, and is present in a quantity between 60% and 90%.

Within the meaning of the present invention, the content quantities in percent may be understood as content quantities according to DIN 1310.

In one particular embodiment, the active ingredient component is present as a gel. Alternatively and/or additionally, the active ingredient component is present as a mixture which includes the organic tooth whitening substance, and which gels only after addition of a solvent. The active ingredient component may thus be present as a powder, for example, and becomes a gel when a predefined quantity of water is added. Suitable gelling agents are known to those skilled in the art, with polysaccharide-based gelling agents being particularly suited.

One particular advantage of the gel is the ease of application to the dentition and the teeth, as well as a corresponding viscosity that improves adhesion and endurance of the tooth whitening preparation on the teeth.

A further advantage of a gel is the good miscibility, for example the mixing of the tooth whitening substance in the active ingredient component that is achievable as a result of the gel network. The viscoelasticity of the gels also makes the application easier and more reliable.

In one alternative or supplementary embodiment, the active ingredient component or the resulting tooth whitening preparation is present as a colloid or as an emulsion. It is thus conceivable, for example, to use glycerin-or fat-based micelles in order to enclose the PAP, i.e., the organic tooth whitening substance, so that it is subsequently present in a dispersion, as a colloid, for example, in the tooth whitening preparation to be used. This may be carried out, for example, using further aids at the point of care, for example by obtaining a fine dispersion of the tooth whitening preparation by means of ultrasound.

A further alternative and/or supplementary embodiment provides a blowing agent, wherein the tooth whitening preparation is formed into a foam and then applied as a foam to the dentition and the teeth. One advantage of a foam is that it exhibits, also under physiological conditions, a viscosity that may facilitate application and endurance over a certain duration of action of the foam on the dentition and the teeth. In addition, the foam enables a largely homogeneous distribution of the tooth whitening substance within the foam, regardless of the pore size.

In one alternative and/or supplementary embodiment, the tooth whitening preparation is present as a liquid in the mixed state. The liquid may be drawn in, for example, via a further absorbent carrier medium, such as a sponge, and by use of the sponge placed on the teeth and the dentition, so that a largely homogenized tooth whitening preparation with the best possible contact with the dentition and the teeth is likewise provided.

Those skilled in the art are familiar with appropriate aids and/or methods to bring the active ingredient component or the resulting tooth whitening preparation into the physical state which facilitates application and provides the tooth whitening substance in a largely homogenized form with the best possible contact with the teeth and the dentition, and may be selected depending on the specific preferred application.

In one particular embodiment, the active ingredient component includes a gelling agent that is selected from the group comprising polysaccharides. The gelling agent is particularly preferably a xanthan.

In one particular embodiment, the activation component contains a base in aqueous solution. The base is particularly preferably an inorganic base selected from the group comprising: ammonia, sodium hydrogen carbonate, calcium hydrogen carbonate, magnesium hydrogen carbonate, sodium hydroxide, potassium hydroxide, calcium hydroxide, magnesium hydroxide, and barium hydroxide. The base is particularly preferably sodium hydroxide. Sodium hydroxide has the advantage, as a strong base, of adjusting the suitable pH in the basic range for the active ingredient component, even in small quantities.

Alternatively or additionally, the activation component includes a mixture of a plurality of the above-mentioned inorganic bases.

Alternatively or additionally, the activation component also contains an inorganic base mixed with an organic base, or as a single base contains an organic base selected from the group comprising: pyridine, methylamine, imidazole, histidine, guanidine, and organic ammonium hydroxides.

In one particular embodiment of the component system according to the invention, the organic tooth whitening substance is selected in such a way that it forms no hydrogen peroxide upon oxidation. Within the meaning of the present invention, it is understood that hydrogen peroxide is also not formed in an intermediate reaction.

In one particular embodiment, the active ingredient component and/or the activation component include(s) at least one indicator. The indicator is preferably designed to indicate a certain pH, in particular a pH range, by means of a color, for example. A change in the pH from a first range into a second range may be indicated by a color change, for example.

In one particularly preferred embodiment, the indicator is designed to display a color change at a pH above 7, preferably at a pH between 8.5 and 9.5. One particular advantage of this approach is that a user can be sure that the pH is in the physiologically acceptable range, and thus that the tooth whitening preparation is ready for application. Thus, for example, an indicator may be added either to the active ingredient component or to the activation component, which in an acidic environment has a certain color; for example, the active ingredient component gives a red coloration or at least imparts a reddish tint, and at a basic pH, preferably at a pH of above 8.5, changes color and for example has a blue color or at least imparts a bluish tint. This has the further advantage that the tooth whitening preparation is present so that it is recognizable by color, which facilitates application of the tooth whitening preparation. Thus, for example, the user sees everywhere that tooth whitening preparation has been applied to the teeth and the dentition. The color change makes the component system according to the invention easier and more reliable in application.

In one particular embodiment, the active ingredient component includes an organic tooth whitening substance in a solution with a pH in a range between 5 and 7. The active ingredient component particularly preferably has a pH in a range of approximately 5.5. In this pH range, the PAP is largely stabilized and has a good storage life. This active ingredient component may preferably be stored at room temperature, but storage under refrigerated conditions at a temperature between 0° and 18° C. is likewise suitable.

In one particular embodiment, the organic tooth whitening substance is a peroxy acid that performs its oxidative action by rapid conversion into a carboxylic acid without the evolution of hydrogen peroxide, and also without hydrogen peroxide arising as an intermediate product. The organic tooth whitening substance is particularly preferably ε-phthalimidoperoxyhexanoic acid (PAP). In use, it would be reacted to form phthalimidohexanoic acid without evolution of hydrogen peroxide, also not as an intermediate product.

In one particular embodiment, the second component container includes a dosing device for dropwise addition of the activation component into the first component container. The activation component is particularly preferably present in a predefined concentration, which in a given quantity is suitable for converting the active ingredient component into a tooth whitening preparation having a physiologically acceptable pH. A dosing device may be designed, for example, to dispense a specified number of drops having a defined drop volume, so that the physiologically acceptable pH may be reliably adjusted.

The second component container particularly preferably includes a mini dropper.

In one particularly preferred embodiment, the first component container is designed to have a volume of the active ingredient component that is suitable for one tooth whitening application. It is thus possible for a fresh tooth whitening preparation to always be provided which can then be directly used by the user. The quantity may also be set to allow multiple passes. Thus, for example to maximize the contact of the teeth and the dentition with the tooth whitening preparation and the active agent therein, i.e., the organic tooth whitening substance, it may be provided that an active ingredient component results in a quantity of tooth whitening preparation that is suitable for several applications.

The first component container is particularly preferably designed for a volume between 4 and 25 mL, preferably 8 to 15 mL, particularly preferably essentially 10 mL, of active ingredient components.

In a further special embodiment, the second component container is designed to bring a plurality of first containers with active ingredient components into the physiologically acceptable pH range as described above. In particular, the second component container may be suited for transferring between 1 and 100 second containers to the physiologically acceptable pH. The second component container may ideally have a volume of essentially 5 mL for this purpose.

The component containers are particularly preferably made of a material that is essentially inert, and that in particular with respect to the materials stored therein does not emit and/or absorb any substances. The component containers are particularly preferably made of glass or plastic.

In one particular embodiment, the active ingredient component and/or activation component and/or the tooth whitening preparation include(s) at least one auxiliary substance selected from the group comprising: desensitizers, remineralizers, gum strengtheners, cosmetic effect agents, and/or whitening boosters.

In one particular embodiment, the desensitizer is an agent that is suitable for minimizing the sensitivity of the gums and dental necks to pain. Such agents are known for use in dentistry and may be selected by the technical specialist as needed. A desensitizer selected from the group comprising potassium nitrate, calcium carbonate, and/or zinc chloride is well suited for the present invention.

In one particular embodiment, a remineralizer is an agent that is suitable for counteracting decalcification (demineralization) of the teeth, for example by providing. lost calcium and/or phosphate ions for the tooth enamel in a usable form. Remineralizers selected from the group comprising hydroxyapatite, fluoride, theobromine, xylitol, and/or tricalcium phosphate are examples of suitable remineralizers for the approach according to the invention.

In one particular embodiment, gum strengtheners are agents that are suitable for alleviating inflammation of the gums and strengthening the gums as a whole. Suitable gum strengtheners for the approach according to the invention are extracts, for example of chamomile, echinacea, peppermint, sage, lemon balm, pepper, mallow, and other botanicals having an anti-inflammatory and/or bacteria-inhibiting effect. Dexpanthenol, pantothenic acid, and/or hyaluronic acid, for example, are/is likewise suitable as strengthening agents.

In one particular embodiment, cosmetic effect agents are additives that achieve a further particular cosmetic effect. Thus, for example, particular color effects with at least a temporary cosmetic action may be achieved by use of pigments, for example. For example, glossy particles that leave a pearlescent sheen or a brilliant shine on the teeth are suitable. Suitable cosmetic preparations are known to those skilled in the art. Likewise suitable are colorants that give the teeth a cosmetic color effect.

In one particular embodiment, a blue light filter containing disodium distyrylbiphenyl disulfonate, for example, is added as a cosmetic effect agent. A filter that filters yellow and red tones is particularly preferably added, so that the teeth have an overall whiter appearance.

In one particular embodiment, the approach according to the invention includes a tooth whitening booster, for example sodium chloride, sodium bicarbonate, or sodium carbonate, that intensifies the action of the active ingredient component and thus enhances the tooth whitening effect.

In one particular embodiment, the component system also includes an application element for applying the tooth whitening preparation to the teeth and/or the dentition, an agitation element for mixing the active ingredient component and the defined quantity of the activation component, and an indicator element for checking the pH of the tooth whitening preparation.

Within the meaning of the present invention, the tooth whitening preparation is a component mixture comprising the active ingredient component and a predefined quantity of the activation component.

The tooth whitening preparation is particularly preferably a component mixture of an active ingredient that includes an imidoperoxycarboxylic acid component as an organic tooth whitening substance, and an alkaline activation component that includes at least one strong base, preferably sodium hydroxide.

In one particular embodiment, the activation component is designed to raise the pH of the active ingredient component to a value between 7 and 10.5, in particular between 8.5 and 9.5, in particular approximately 5.5 to approximately 9.5, using a defined quantity of an alkaline activation component. The activation component particularly preferably includes sodium hydroxide solution for this purpose.

The active ingredient component and the activation component may include further auxiliary substances that may be used to ensure physical and/or chemical properties of the component in question. Thus, for example, buffers or complexing agents may be provided which keep the pH in the components in question to a certain value, or which stabilize substances and at least temporarily protect them from undergoing reaction.

For those skilled in the art, it is self-evident that in an embodiment of the teaching according to the invention, a component system may have any given combination of the above-described features, provided that they are not mutually exclusive.

A corresponding method as well as further aspects associated with the teaching according to the invention are described below. From this description, a person skilled in the art may also derive further structural features for a component system according to the invention.

A further aspect of the present invention relates to a stabilized active ingredient component for use in a component system, as described at the outset, for producing an activated physiologically acceptable tooth whitening preparation. The stabilized active ingredient component includes at least one organic tooth whitening substance that is an oxidation agent, at least in a basic environment. The stabilized active ingredient component also includes a carrier for stabilizing an essentially homogeneous mixture of the organic tooth whitening substance with a pH lower than 7, in particular approximately 5.5.

In one preferred embodiment, the stabilized active ingredient component is prepared in such a way that essentially the entire prepared quantity is consumed in one tooth whitening application. The active ingredient component may thus be used as an expendable material. In contrast, the activation component may be used to activate a plurality of active ingredient components or to make a conversion into a physiologically acceptable pH range. Accordingly, the active ingredient component is designed so that its volume essentially completely covers an average set of teeth with a layer of the tooth whitening preparation.

Preferably only the front side of the teeth is covered with such a layer. To ensure that a certain exposure period for the tooth whitening preparation can be maintained, various aids may be used, for example a lip retractor for keeping the lips away from the teeth and/or a suction apparatus for aspirating saliva.

In one particular embodiment, the stabilized active ingredient component is present as a solution, a dispersion, or a colloid at room temperature. It is particularly preferably present as a dispersion, for example in a gel.

In one particular embodiment, the stabilized active ingredient component includes at least one auxiliary substance from the group comprising: water, thickeners, in particular at least one polysaccharide, alcohols, gelators, emulsifiers, binders, film-forming agents, and/or stabilizers.

A further aspect of the present invention relates to a method for preparing an activated physiologically acceptable tooth whitening preparation.

The method includes the step of providing an active ingredient component that includes an organic tooth whitening substance.

The method further includes the step of providing an activation component, in particular an alkaline activation component, for adjusting a physiologically acceptable activated pH of the active ingredient component.

Lastly, the method according to the invention includes the step of adding a defined quantity of the activation component to the active ingredient component and mixing these two components to obtain the tooth whitening preparation.

The active ingredient component is particularly preferably designed in such a way that at a basic pH it has a high oxidation capability. The method according to the invention is preferably used for cosmetic tooth whitening, i.e., to obtain visible whitening and a more intense white coloration of the teeth. For this purpose, the active ingredient component may be used with the organic tooth whitening substance to oxidize staining substances and thus neutralize the color. In particular, the consumption of coffee, tea, tobacco, as well as certain medications may result in the teeth acquiring a yellowish tint, regardless of genetic disposition.

By use of the method according to the invention it is possible to lighten this shade along a color scale. It is likewise possible to further lighten a shade that is darker than desired due to genetic reasons. Medical use of the approach according to the invention is conceivable in particular applications, for example when discoloration of the teeth is present due to medical reasons. This may also have a genetic origin or may be due, for example, to a particular treatment, for example radiation therapy or taking certain medications such as tetracyclines, which are known to cause discoloration of the teeth.

In one particular embodiment, the method according to the invention encompasses impingement of the teeth with light, preferably light in the spectral range between 400 nm and 500 nm. The impingement of the teeth with light particularly preferably takes place while the tooth whitening preparation is applied to the teeth, i.e., during the exposure period.

A further aspect of the present invention is the use of a component system, mentioned at the outset, for preparing a physiologically acceptable tooth whitening preparation for medical treatment of discoloration of the teeth. An active ingredient component having a minimum concentration of PAP in the range of 10 to 20 wt %, among other substances, is used for this purpose. It is particularly preferred to use a concentration of PAP in the range between 12 and 14% in the method according to the invention.

The present invention is explained in greater detail below based on specific examples and embodiments. Further advantageous combinations of features result for those skilled in the art. These special embodiments and examples are not to be construed as limiting for carrying out the inventive teaching of the present invention.

### DESCRIPTION OF THE FIGURES

Exemplary embodiments of the invention are described with reference to the following figures.

In the figures:

FIG. 1 shows the schematic sequence of a: method according to the invention, and

FIG. 2 shows an example of preparation of the component system according to the invention.

### IMPLEMENTATION OF THE INVENTION

### Example 1

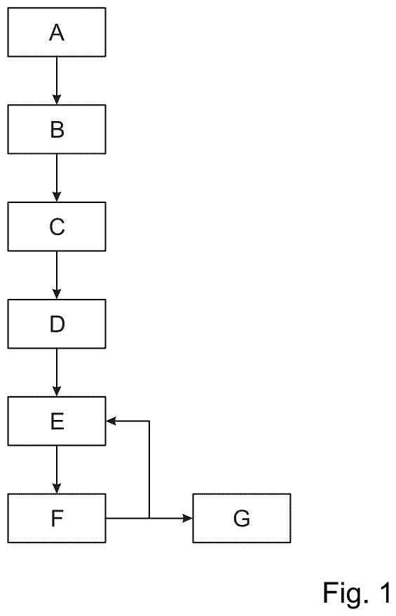

In the present example, an application as schematically illustrated in FIG. 1 is described by way of example, and as may be used, for example, in training of technical personnel or as specific operating instructions for the use of a component system according to the invention.

In the present example, the active ingredient component is present as a gel, and a liquid activation component is prepared separately in a container. The active ingredient component is present in a quantity that is sufficient to perform a complete treatment of the teeth. The dentition may be treated essentially completely, but preferably in the area of the front row of teeth. An example application within the meaning of the invention may be, for example, application to the visible area of the teeth by prompting the patient to smile prior to the treatment, so that it can be seen how wide the smile is, and whether the second premolars, for example, are also visible and must also be treated. However, for each jaw generally the labial surfaces of eight teeth are whitened, namely, the outer faces (labial, facing the lips) of teeth 14-24 in the upper jaw and teeth 34-44 in the lower jaw. For preparation, to an active ingredient component gel of approximately 10-15 mL (preferably approximately 10 mL), a quantity of two drops of the activation component is added to the active ingredient component. In professional use, also in the field of pharmacy, for example, the quantity of drops is a defined variable referred to as “gutta” (gtt). In precise droppers the dispensed quantity is approximately 0.05 mL. In this example, the concentration of activation component according to the invention is selected so that a quantity of n times 0.05 mL is suitable for converting the active ingredient component to the desired physiologically acceptable pH. In the present example, a quantity of 4 drops, i.e., a total of 0.2 mL (4 gtt), is sufficient. Specific examples of mixture compositions are described in the further examples below.

The active ingredient component is blended as homogeneously as possible using a medical spatula. A certain mixing time is ideally specified, which may be checked with a timer, for example. Within the meaning of the present example, a mixing time of 60 seconds has been found to be suitable. For certainty, the pH of the gel is measured using a pH test strip.

In this particular exemplary embodiment the pH is preferably between 8.5 and 9.5. As an alternative to measurement using a pH strip, the active ingredient component may already be provided with an indicator. Suitable chemical indicators that exhibit a certain color at a given pH are known to those skilled in the art.

Use of a pH test strip, an indicator, or a pH electrode ensures that the pH is in this defined range. When it is determined that the pH is in the suitable range, a series of application cycles is carried out over a certain time period.

The application cycles may each take place over a period of between 15 and 30 minutes. For the present example, a total application period of 60 minutes has been found to be particularly suitable, in which two to four application cycles, each lasting 15 to 30 minutes, may be carried out. Between the application cycles, the used active ingredient component is removed from the oral cavity by rinsing. For the rinsing, an appropriate rinse solution may be provided which is suitable for breaking up the polysaccharides of the active ingredient gel and thus preventing the active ingredient component from adhering to the teeth. However, rinsing with water or a simple tooth rinse solution, for example an NaCl solution, is likewise productive.

Rinsing is ideally performed for a certain time period; in the present example, a period of 60 seconds has been shown to be ideal.

In order to ensure during the treatment that the active ingredient component stays on the teeth, the mouth is held open by means of a cheek retractor.

The method according to the invention is schematically illustrated in FIG. 1. The method steps may take place according to the example as follows: A) providing the active ingredient component, B) filling the active ingredient component, for example 10 g of the active ingredient component, into a crucible or a dish, C) adding an activation component, such as a predefined quantity of 25% sodium hydroxide solution, for example 0.29 to 0.37 g of a 25% sodium hydroxide solution, for example by use of a dropper, and subsequently D) mixing or stirring over a period of 60 s. The resulting physiologically acceptable tooth whitening preparation is applied to the teeth in a step E), and acts over a period of 10-20 min. After the exposure period, F) rinsing is performed and the application/exposure from step E) is repeated two to five, preferably four, times until G) final rinsing is performed.

### Example 2

Stabilized active ingredient component for use with a component system according to the invention:

| Ingredient | wt % |
|---|---|
| H2O (distilled water) | 72% |
| 6-(Phthalimido)peroxyhexanoic acid | 13.8% |
| Glycerin | 9.9% |
| Xanthan | 3.3% |
| Sodium citrate | 0.3% |
| PVM/MA copolymer | 0.3% |
| Sodium hydroxide | 0.3% |
| C12-15 Pareth-3 | 0.1% |

### Example 3

Alternative stabilized active ingredient component for use with the component system according to the invention:

| Ingredient | wt % |
|---|---|
| H2O (distilled water) | 73.6% |
| 6-(Phthalimido)peroxyhexanoic acid | 12% |
| Glycerin | 10% |
| Xanthan | 3.2% |
| Sodium citrate | 0.4% |
| PVM/MA copolymer | 0.4% |
| Sodium hydroxide | 0.3% |
| C12-15 Pareth-3 | 0.1% |

### Example 4

An activation component according to the invention for use in a component system as described at the outset may be an aqueous solution of a strong base.

In the present example, the activation component includes as an essential component an alkaline substance, in this case a strong base in the form of a 25% or 50% sodium hydroxide solution in water. Depending on the starting concentration of the base in the activation component, a different number of drops may be necessary to adjust to the desired pH.

With 25% NaOH, 3-4 drops are sufficient to raise the pH of the active ingredient component from 5.5 to 8.5-9.5.

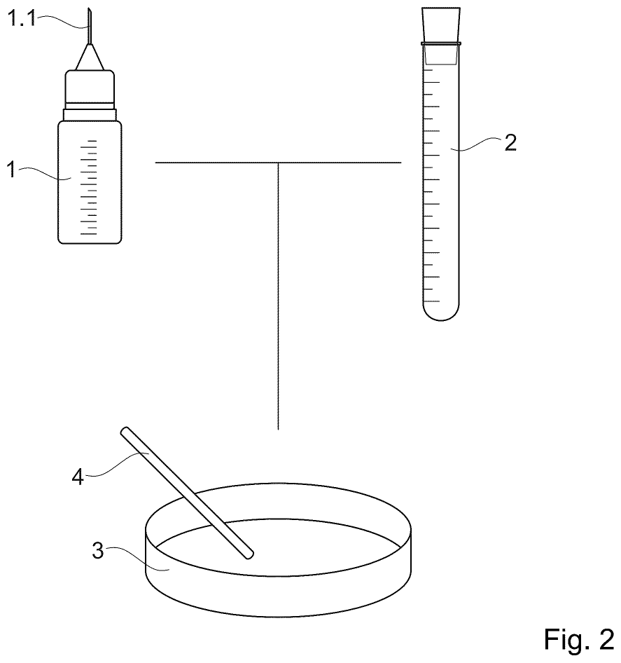

FIG. 2 shows an example of how a component system according to the present invention may be prepared, and how it is converted into a tooth whitening preparation. The component system includes an active ingredient component that is contained in a first component container 2, and an activation component that is contained in a second component container 1. The active ingredient component is present in the first component container 2 in a quantity that is sufficient for four application cycles, i.e., approximately 10 g. The activation component may be present in a quantity of 5 mL. The second component container 1 correspondingly also includes a dosing tip 1.1 for dropwise dispensing of the activation component. In the present example, the dosing tip 1.1 is selected so that a quantity of 0.29 to 0.37 g is to be dispensed with 3 to 4 drops. This quantity is selected in such a way that for a 25% NaOH solution in water as activation component, a pH of 8.5 to 9.5 is set in the resulting tooth whitening preparation. The two components are admixed in a dish 3 by use of a stirring rod.

In a further special embodiment, a pH of 9.6 to 9.9, particularly preferably a pH of 9.9, is set. In this special embodiment, a quantity that is sufficient for four application cycles, for example, i.e., approximately 10 g, is divided in advance into four portions of approximately 2.5 g each, and before each application cycle each portion is set to the desired pH, in particular a pH of 9.9, using 3 to 4 drops of 25% NaOH solution in each case.

In one particular embodiment, the first component container 2 is designed as a crucible and may replace the dish, or may be used directly for mixing the tooth whitening preparation.

The present invention provides a system for reliably applying, in a point-of-care situation, an active ingredient component containing an organic tooth whitening substance, which per se has a stable pH in an aqueous solution, is physiologically harmless, and can have no adverse effects for treatment of the teeth (the stabilized pH of PAP in aqueous solution is between 3.3 and 5.5).

For this purpose, a component system is provided in which the active ingredient component in its stable form is prepared separately from an activation component, which brings this active ingredient component to a physiologically acceptable pH and develops the full oxidation potential of the organic tooth whitening substance.

By use of the component system according to the invention, a tooth whitening preparation is provided that has a high efficiency, can be used with a sufficient concentration of organic tooth whitening substance, and is reliable to use. The preparation is also advantageous, it being possible to select the activation component in such a way that even a small quantity in the amount of a few drops may be sufficient to set the required pH, and thus to prepare the activation component so that the activation component may be used multiple times. At the same time, the active ingredient component may be prepared in a quantity such that it is adequate for exactly one application or a series of successive application cycles. The oxidation potential is subsequently depleted, and the active ingredient component is freshly activated in each case as a tooth whitening preparation prior to each use or each series of successive application cycles.

Due to the stabilization of the active ingredient component, it is also possible to plan for the inclusion of further auxiliary substances, either in the activation component or directly in the active ingredient component, so that these auxiliary substances likewise develop their desired effect. Thus, for example, supplementary tooth whitening substances such as sodium carbonate may be provided which may also be used as a base for activating the active ingredient component. Other active ingredients for cosmetic and/or medical effects may likewise be provided within the scope of the present invention.

The product can thus meet stringent safety requirements, and for point-of-care applications offers an ideal alternative with fewer side effects compared to the conventional hydrogen peroxide-based tooth whitening products.

1. A component system for preparing a physiologically acceptable tooth whitening preparation, comprising:
a. an active ingredient component, in particular with a pH below 7, that includes an organic tooth whitening substance; and
b. an activation component, in particular an alkaline activation component, for adjusting a physiologically acceptable pH of the active ingredient component in a range between 7 and 10.5, in particular between 8.5 and 9.5.

2. The component system according to claim 1, wherein the activation component contains a base in solution with a base constant pkb of 4 maximum.

3. The component system according to claim 1, wherein the active ingredient component contains at least one caproic acid as an organic tooth whitening substance, in particular a peroxy acid that is dissolved in an aqueous solution, and that in particular is an imidoperoxycarboxylic acid, preferably PAP.

4. The component system according to claim 1, wherein the organic tooth whitening substance is present in the active ingredient component in a concentration of at least 7%, in particular in a concentration between 10% and 30%, in particular between 10% and 20%.

5. The component system according to claim 1, wherein the active ingredient component is present as a gel.

6. The component system according to claim 1, wherein the activation component contains a base in aqueous solution, selected from the group of inorganic bases, in particular from the group comprising: ammonia, sodium hydrogen carbonate, sodium hydroxide, potassium hydroxide, calcium hydroxide, magnesium hydroxide, and barium hydroxide.

7. The component system according to claim 1, wherein the active ingredient component including an organic tooth whitening substance has a pH between 5 and 7, in particular 5.5.

8. The component system according to claim 1, wherein the active ingredient component is in a first component container and the activation component is in a second component container.

9. The component system according to claim 8, wherein the second component container includes a dosing device for dropwise addition of the activation component into the first component container.

10. The component system according to claim 1, wherein the component system further includes:
a. an application element for applying a component mixture to the teeth, and/or
b. an agitation element for mixing the active ingredient component with a defined quantity of the activation component, and/or
C. an indicator element for checking the pH of a component mixture.

11. The component system according to claim 1, wherein the activation component is designed to raise the pH of the active ingredient component to a value between 7 and 10.5, in particular between 8.5 and 9.5, in particular approximately 5.5 to approximately 9.5, using a defined quantity of an alkaline activation component.

12. A stabilized active ingredient component for use in a component system, in particular a component system according to claim 1, for preparing an activated physiologically acceptable tooth whitening preparation, including:
a. an organic tooth whitening substance that is an oxidation agent in a basic environment; and
b. a carrier for stabilizing an essentially homogeneous mixture of the organic tooth whitening substance with a pH lower than 7, in particular approximately 5.5.

13. The stabilized active ingredient component according to claim 12, wherein the stabilized active ingredient component is present as a solution, a dispersion, or a colloid at room temperature.

14. The stabilized active ingredient component according to claim 12, including at least one auxiliary substance from the group comprising: water, thickeners, in particular polysaccharides, alcohols, chelators, emulsifiers, binders, film-forming agents, and stabilizers.

15. A method for preparing an activated physiologically acceptable tooth whitening preparation, comprising the steps:
a. providing an active ingredient component that includes an organic tooth whitening substance;
b. providing an activation component, in particular an alkaline activation component, for adjusting a physiologically acceptable activated pH of the active ingredient component; and
c. adding a defined quantity of the activation component to the active ingredient component and mixing the two components.
